(19) United States
(12) Patent Application Publication
(10) Pub. No.: US 2015/0018967 A1
(43) Pub. Date: Jan. 15, 2015

(54) Duodenum Endothelium Membrane Made From Degradable Biocompatible Materials and Application Thereof

(71) Applicant: Ping WAN, Shanghai (CN)

(72) Inventor: Ping Wan, Shanghai (CN)

(21) Appl. No.: 14/379,735

(22) PCT Filed: Apr. 27, 2013
(86) PCT No.: PCT/CN2013/074836

§ 371 Date: Aug. 19, 2014

(30) Foreign Application Priority Data
May 5, 2012 (CN) 201220197782.9

(51) Int. Cl. A61F 5/00; A61F 2/04

(52) U.S. Cl. CPC A61F 5/0076; A61F 2/04; A61F 2002/045; A61F 2210/0004; A61F 2210/0057; A61F 2210/0014
USPC 623/23.68

(57) ABSTRACT

This invention provides a duodenum endothelium membrane made of degradable shape-memory biocompatible materials. The endothelium membrane planted in the duodenum can divide chyme and bile-pancreatic juice, avoiding direct digestion, absorption, and metabolism of gastric effluent in the duodenum. After being implanted, the endothelium membrane is stable and difficult to slip and incarcerate, and is gradually degraded through two months to five years, avoiding complicated operation and organ tissue injury when removing later, and slowing down the “rebound” effect after complete demolition of the original barrier in an instant. The endothelium membrane is prepared into the medical device free of removing and treating obesity and diabetes. The endothelium membrane can avoid the deflation of the tubular portion induced by gastrointestinal peristalsis and gap increase between the endothelium membrane of the tubular portion and inner wall of the duodenum further to avoid regurgitation of jejunum contents.

### BACKGROUND OF THE INVENTION

1. Field of the Invention

This invention relates to a built-in degradable medical device for a digestive tract and, more particularly, to a duodenum endothelium membrane capable of treating obesity and diabetes.

2. Description of the Related Art

“In recent years, through the analysis on operation result of obesity abroad, it is found that the weight of obesity people is remarkably reduced, and the condition of their concurrent type 2 diabetes is unexpectedly relieved after they are subjected to the gastrointestinal surgery, and there is also similar report in domestic.” (Surgical treatment of diabetes expert consensus. Chinese Journal of Diabetes, 2011, 3 (3):205-208)

“The weight loss surgery shall be considered for suitable patients early, to prevent the occurrence of the serious complications of diabetes” (Expert Interpretation: Weight Loss Surgery for Diabetes Treatment. Diabetes World: Digest, 2011, 10:51).

“The surgical treatment brings the new treatment means to the obese type 2 diabetic patients”, “In March 2011, the international diabetes federation (IDF) firstly stated in the second international type 2 diabetes interventional therapy meeting held in New York, that it thought that the gastric bypass surgery can be applicable to treat the obese type 2 diabetic patients, and can reduce the occurrence and development of diabetic chronic complications.” (Du Tao, He Xiao. Surgical treatment of diabetes need to regulate and multidisciplinary collaboration—Interview with director of Capital Medical University, Professor of Endocrinology, Beijing Tiantan Hospital, Zhong Li Yong, China Medical Sciences, 2011, 1 (22): 1-2)

“Gastric bypass surgery is metabolic surgery. Abroad, the U.S. government are included for actively promoting the development of the metabolic surgery, because patients can benefit from this surgery largely, after the operation, it does not have to inject insulin, and also, there is no need to take multiple medications, then, blood sugar problems can be solved, and high blood pressure, obesity, lipid disorders and other complications of diabetes can show significant improvement. A comprehensive analysis of 22094 cases of patients showed that: 84% of type 2 diabetes are completely reversed after surgery, and most patients

stopped oral medication or insulin therapy before discharge.” (Feng Yisheng. Golden autumn meeting, Multidisciplinary Experts' “Consultation” on Diabetes Surgery—“Surgical treatment of diabetes Heaven 2011 Conference” held in Beijing. China Medical Sciences, 2011, 1 (21):3-5)

“In addition, the study on health economics for surgical treatment of obesity found that surgical treatment can achieve a better balance between income and expenses, so as to reduce economic burden on obese diabetic patients themselves and society.” (Surgical treatment of diabetes expert consensus. Chinese Journal of Diabetes, 2011, 3 (3):205-208)

But the above ‘gastric bypass surgery’ has clinical risks, such as death, bowel obstruction, anastomotic leak, pulmonary embolism, deep vein thrombosis, portal vein injury, and respiratory system diseases. (Surgical treatment of diabetes expert consensus. Chinese Journal of Diabetes, 2011, 3 (3):205-208)

Thus, in the body, the obesity and diabetes are treated by implanting the duodenum endothelium membrane in the duodenum, so as to substitute the above-mentioned “gastric bypass surgery”. The study of the duodenum endothelium membrane, which following the evaluation and guidance principle of medical device biocompatibility and standard test method (such as ISO 10993-1992) formulated by the international standard organization (ISO) and national standard on biological evaluation of medical devices GB/T16886, still has the applied significance.

The “non-toxic outer tube” is not equivalent to the biocompatibility of the broader concept in the patent invention “duodenal tube and conveyor thereof” (the application date is Apr. 9, 2010, and the date of authorization proclamation is Jan. 11, 2012) in the prior art, and the biocompatibility may be subjected to many others such as an allergic test, an intracutaneous stimulation test and the like, except non-toxicity (an acute systemic toxicity test). Even the “non-toxicity” represents the “biocompatibility”, but “the outer surface of its metal skeleton is covered by the outer tube, so as to hinder the damage of the metal component to the duodenal mucosa”, indicating that the metal component in the prior art has the damage to the organism, and “the outer tube is coated outside the metal skeleton” and “the outer tube is connected with the metal skeleton through adhesive”, indicating that the metal skeleton “which has the damage to the organism” in the prior art is not entrapped in the middle of the outer tube wall, and is exposed in the cavity and directly contacted with chyme. Moreover, the biocompatibility of the material directly contacted with chyme cannot be ignored, just like the biocompatibility of the material contacted with blood must be verified. In the utility model patent “duodenum-jejunum built-in sleeve” (the application date is Dec. 6, 2010, and the date of authorization proclamation is Sep. 28, 2011) in the prior art, only “the close mouth line is made of fluoropolymer line which is provided with good biocompatibility and corrosion resistance”.

The “metal ring with internal support” (the patent invention “duodenal tube and conveyor thereof”) in the prior art internally supports the fixed tube for long dynamic peristalsis and soft bowel of compensatory expansion only by relying on the disposable static “expansion” of “memory alloy metals”, which is not only difficult to achieve, and but also has slipped and incarcerated risks.

In the prior art (the utility model patent “duodenum-jejunum built-in sleeve”), to solve the problem of fixation, the hollow metal tube is made into a spiky fixed jaw, “the circular tube part of the metal tube” is also movably “sleeved on the metal wire of the ring support”, and fixed tightly. Then, in order to solve the removal problem in the future, the close mouth line is also designed in the prior art (the utility model patent “duodenum-jejunum built-in sleeve”). The close mouth line is “placed at the top of the ring support”, “which can surround the upper opening for one circle, and can also surround the upper opening for many circles”. After being implanted in the body, in particular, the spiky fixed jaw made of the hollow metal tube is penetrated into the inner wall of duodenal bulb, and with the gastrointestinal peristalsis, continuous looseness, exudation and adhesion, the spiky fixed jaw made of the hollow metal tube has been firmly (focused on the firmness in the prior art) embedded in the intestinal tissue (when the foreign body is penetrated into the intestinal tissue, and it may stimulate the growth of partial intestinal tissue in case of not being digested or discharged, generally, it is closely adhered after three weeks, and this is common general knowledge of digestive surgical technician) of creep growth when removing in the second “surgery” later.

The flexible hose in the utility model of the prior art “duodenum-jejunum built-in sleeve” is elastic but not plastic (non-shape memory material), which is easy to lead the deflation of the tubular portion induced by gastrointestinal peristalsis and gap increase between the endothelium membrane of the tubular portion and the inner wall of the duodenum and then leading the regurgitation of jejunum contents.

Even without considering the problem for the complicated operation process of the second “surgery” and organ tissue injury when removing in the future, after removing in the second “surgery” later in the prior art (the patent invention “duodenal tube and conveyor thereof”, and the utility model patent “duodenum jejunum built-in sleeve”), the original division barrier between the chyme and bile-pancreatic juice is completely demolished in an instant, the chyme and bile-pancreatic juice are not divided in an instant, and the direct digestion, absorption and metabolism of gastric effluent in duodenum is recovered in an instant. Therefore, it is difficult to completely avoid the “rebound” problem after the complete demolition of the original barrier in an instant.

### BRIEF SUMMARY OF THE INVENTION

### Technical Problems Solved by the Invention

The “non-toxic outer tube” is not equivalent to the biocompatibility of broader concept in the invention patent “duodenal tube and conveyor thereof” (the application date is Apr. 9, 2010, and the date of authorization proclamation is Jan. 11, 2012) in the prior art, and the biocompatibility may be subjected to many other, such as an allergic test, an intracutaneous stimulation test and the like, except non-toxicity (an acute systemic toxicity test). Even the “non-toxicity” represents the “biocompatibility”, but “the outer surface of its metal skeleton is covered by the outer tube, so as to hinder the damage of metal component to the duodenal mucosa”, indicating that the metal component in the prior art has the damage to the organism, and “the outer tube is coated outside the metal skeleton” and “the outer tube is connected with the metal skeleton through adhesive”, indicating that the metal skeleton “which has the damage to the organism” in the prior art is not entrapped in the middle of the outer tube wall, and is exposed in the cavity and directly contacted with chyme. Moreover, the biocompatibility of material directly contacted with chyme cannot be ignored, just like the biocompatibility of material contacted with blood must be verified. In the utility model patent “duodenum-jejunum built-in sleeve” (the application date is Dec. 6, 2010, and the date of authorization proclamation is Sep. 28, 2011) in the prior art, only “the close mouth line is made of the fluoropolymer line which is provided with good biocompatibility and corrosion resistance” (the invention does not need to remove in the future, therefore, the close mouth line is not arranged). All components of the duodenum endothelium membrane provided by this invention are made of biocompatible materials, thus solving the biocompatible problem of material implanted in the body and weakening the host response generated by implanting in the body.

The “metal ring with internal support” (the patent invention “duodenal tube and conveyor thereof”) in the prior art internally supports the fixed tube for long dynamic peristalsis and soft bowel of compensatory expansion only by relying on the disposable static “expansion” of “memory alloy metals”, which is not only difficult to achieve, and but also has slipped and incarcerated risks. The stretch yarn with the anchor hook is surrounded outside the ampulla of the duodenum endothelium membrane in this invention, which can solve this problem.

In the prior art (the utility model patent “duodenum-jejunum built-in sleeve”), to solve the problem of fixation, the hollow metal tube is made into a spiky fixed jaw, “the circular tube part of metal tube” is also movably “sleeved on the metal wire of the ring support”, and fixed tightly. Then, in order to solve the removal problem in the future, the close mouth line is also designed in the prior art (the utility model patent “duodenum-jejunum built-in sleeve”). The close mouth line is “placed at the top of the ring support”, “which can surround the upper opening for one circle, and can also surround the upper opening for many circles”, but after being implanted in the body, in particular, the spiky fixed jaw made of hollow metal tube is penetrated into the inner wall of duodenal bulb, and with the gastrointestinal peristalsis, continuous looseness, exudation and adhesion, the spiky fixed jaw made of the hollow metal tube has been firmly (focused on the firmness in the prior art) embedded in the intestinal tissue (when the foreign body is penetrated into the intestinal tissue, and it may stimulate the growth of partial intestinal tissue in case of not being digested or discharged, generally, it is closely adhered after three weeks, and this is common general knowledge of digestive surgical technician) of creep growth when removing in the second “surgery” later. However, the duodenum endothelium membrane provided by this invention can be gradually degraded in the body through two months to five years after being implanted in the body, thus solving the problem of complicated operation process and organ tissue injury when removing in the body, and avoiding the organ tissue injury when removing in the body later.

The flexible hose in the utility model of the prior art “duodenum-jejunum built-in sleeve” is elastic but not plastic (non-shape memory material). However, the tubular portion of the elastic plastic model in this invention is made of elastic shape memory materials. Therefore, when transferring the intestinal peristalsis, the problem on the deflation of the tubular portion induced by gastrointestinal peristalsis and gap increase between the endothelium membrane of the tubular portion and inner wall of the duodenum and then the regurgitation of jejunum contents is solved.

Even without considering the problem for the complicated operation process of the second “surgery” and organ tissue injury when removing in the future, after removing in the second “surgery” later in the prior art (the invention patent “duodenal tube and conveyor thereof”, and the utility model patent “duodenum-jejunum built-in sleeve”), the original division barrier between the chyme and bile-pancreatic juice is completely demolished in an instant, the chyme and bile-pancreatic juice are not divided in an instant, and the direct digestion, absorption and metabolism of gastric effluent in duodenum is recovered in an instant. However, the duodenum endothelium membrane in this invention can be gradually degraded in the body through two months to five years after being implanted in the body, thus solving the problem the “rebound” problem after the complete demolition of the original barrier in an instant, and slowing down the rebound effect. Moreover, the duodenum endothelium membrane can be prepared into a medical device capable of preventing falling off, being free of removing, restraining bounce, reducing the injury, and treating obesity and diabetes.

### Technical Scheme of the Invention

All components of the duodenum endothelium membrane are made of the shape-memory degradable biocompatible materials. The shape-memory degradable biocompatible materials can be obtained through polyurethane blended membrane (which can be obtained through synthesizing polyurethane on its main chain or side chain with hydroxyl-contained phosphorylcholine monomer, and can also be obtained through modifying polyurethane with surface grafting phosphorylcholine monomer) modified by phosphorylcholine materials and chitosan polyurethane blended membrane. The shape-memory degradable biocompatible materials can take polycaprolactone (PCL), polylactic acid (PLA), polyglycollide (PGA) and polyglycollide-lactide (PLGA), polytetramethylene ether glycol (PTMG) and copolymer thereof and other polyesters, having a good slow-release function and safe degradation product, as the soft segment, and take 2, 6-diisocyanate (LDI), 1, 4-diisocyanate (BDI), and hexamethylene diisocyanate (HDI) as well as copolymer thereof as the hard segment. Aromatic diisocyanate is reacted with polyester or polyether having certain molecular weight to generate prepolymer of carbamic acid, and then subjected to the chain extension with polyols or polyamine and others to generate polyurethane with a block structure. The shape-memory degradable biocompatible materials in the invention can be prepared with the different aggregation states and thermal behaviors of the soft segment (polyester or polyether) and the hard segment (carbamate) of segmented polyurethane. The soft segment part is in a reversible phase, and when stretching at the certain heat degree, the curly molecular chain is stretched in the exogenic action and oriented, and then, cooled in the keeping of exogenic action; and the soft segment is crystallized or vitrified to freeze the stress, and while the intrinsic heat at the certain temperature, the stress frozen is gradually released and gradually recovered the deformation in the effect of polyurethane elasticity. The hard segment part is a physical crosslinking point; and at certain heat degree, the deformation can be carried out in the interval above the melting point of the soft segment and below the melting point of the hard segment through giving the modulus and strength to the polyurethane. Through adjusting the content or category of soft segment and hard segment parts, the shape-memory degradable biocompatible materials having the required degradation rate, elasticity modulus, memory temperature, crystallinity, tensile strength, Young modulus and other main properties can be plasticized.

The duodenum endothelium membrane may include an ampulla and a tubular portion. The stretch yarn with an anchor hook is surrounded outside the ampulla.

The diameter and length of the tubular portion are matched with the duodenum and jejunum of different crowds, of which the diameter is 10-60 mm. The length is matched with the duodenum and can be extended to a section of the jejunum continued with the duodenum, and the length is 80-700 mm. The thickness of the endothelium membrane of the tubular portion is 0.005 mm-1mm.

The tubular portion of the elastic plastic model is made of the elastic shape memory material. Therefore, when the intestinal peristalsis is transferred, the problem on the deflation of the tubular portion induced by gastrointestinal peristalsis and gap increase between the endothelium membrane of the tubular portion and the inner wall of the duodenum and then the regurgitation of jejunum contents can be avoided.

The ampulla is the part of trumpet-shaped consecutive tubular portion, as well columnar, spherical columnar, and drum-shaped. The thickness of the endothelium membrane of the ampulla is 0.005 mm-1 mm, and its height is 6 mm-100 mm. The trumpet-shaped consecutive tubular portion is progressive open acute angle, of which the angle is 5 degrees to 45 degrees. The thickness, height, and angle of the ampulla are matched with the different crowds.

The stretch yarn provided with the anchor hook is surrounded outside the ampulla of the consecutive tubular portion, of which the yarn diameter is 0.05 mm-1 mm; and the stretch yarn and ampulla are integrated as a whole. The explanate stretch yarn can be rectilinear, as well V-shaped, U-shaped, O-shaped, grid-shaped, and parabolic. The explanate stretch yarn with various shapes can be surrounded the ampulla with a single circle or many circles, and each circle of multi-circle surrounding can be separated without closing. The stretch yarn is attached with an anchor hook every a distance, of which the distance is 5 mm-20 mm, and the anchor hook is faced towards the end of the tubular portion, of which the included angle is 5 degrees-75 degrees, and the length is 0.3 mm-10 mm.

The duodenum endothelium membrane can be gradually degraded in the body through two months to five years after being implanted, and the degradation product and soluble substance are safe to the human body, without removal, thereby, avoiding the tissue injury, and slowing down the rebound effect after the complete demolition of the original barrier in an instant by gradually degrading.

The duodenum endothelium membrane is soft, smooth, elastic, free of acute systemic response, chronic systemic response, acute local response and chronic local response, and has good histocompatibility.

The ampulla and tubular portion of the duodenum endothelium membrane can be converged into a spherical or capsular form, or folded into a spherical or capsular form in vitro together.

The duodenum endothelium membrane can be sent into the duodenum through the upper digestive tract in the assistance of endoscope and X-ray fluoroscopy, and gradually unfolded under the intra-intestinal memory temperature. The ampulla of the endothelium membrane is located above the duodenum, and the lower edge of the ampulla is located at the approximate stomachus pyloricus side of duodenal papilla and auxiliary papilla (or papillula), which does not hinder the liquid in the bile duct and pancreatic duct to enter into the enteric cavity. The tubular portion of the endothelium membrane is located at the descending part, horizontal part and ascending part of consecutive duodenum of the superior part of the duodenum, and the extended tubular portion is located at the ascending part of duodenum and consecutive jejunum section.

The anchor hook on the stretch yarn surrounded outside the ampulla of the duodenum endothelium membrane is naturally inserted into the intestinal mucosa of duodenal bulb cavity section and tela submucosa, so that the duodenum endothelium membrane is fixed in the duodenum.

The duodenum endothelium membrane divides the chyme and bile-pancreatic juice in the body, so as to avoid the direct digestion, absorption and metabolism of gastric effluent in the duodenum. Therefore, it can be prepared into the medical device capable of treating the obesity and diabetes without removing from the body.

### Beneficial Effects of the Invention

A duodenum endothelium membrane made of shape-memory degradable biocompatible materials is provided. The endothelium membrane is implanted into the duodenum, to divide the chyme and bile-pancreatic juice in the body, thereby, avoiding the direct digestion, absorption and metabolism of gastric effluent in the duodenum. Compared with the prior art (the patent invention “duodenal tube and conveyor thereof”, and the utility model patent “duodenum-jejunum built-in sleeve”), the duodenum endothelium membrane provided by this invention is free of anaphylaxis, toxic response, anaphylaxis, mutagenicity, carcinogenicity, teratogenicity and inhibition of cell activation, and has good histocompatibility, thereby, weakening the host response generated by implanting in the body. Compared with the prior art (the patent invention “duodenal tube and conveyor thereof”, and the utility model patent “duodenum-jejunum built-in sleeve”), after being implanted in the body, the duodenum endothelium membrane provided by the invention is stable and difficult to slip and incarcerate, and can be gradually degraded through two months to five years in the body, thereby, avoiding the complicated operation and organ tissue injury when removing in the body later, and slowing down the “rebound” effect after the complete demolition of the original barrier in an instant by gradually degrading. Moreover, the duodenum endothelium membrane can be prepared into the medical device capable of being free of removing and treating obesity and diabetes. Compared with the prior art (the patent invention “duodenal tube and conveyor thereof”, and the utility model patent “duodenum-jejunum built-in sleeve”), the duodenum endothelium membrane provided by the invention can avoid the deflation of the tubular portion induced by gastrointestinal peristalsis and gap increase between the endothelium membrane of the tubular portion and inner wall of the duodenum further to avoid the regurgitation of jejunum contents. The medical device prepared for treating the obesity and diabetes can prevent falling off, being free of removing, restraining bounce, and reducing the injury.

### BRIEF DESCRIPTION OF THE DRAWINGS

FIG. 1 is a schematic diagram according to the embodiments of the invention.

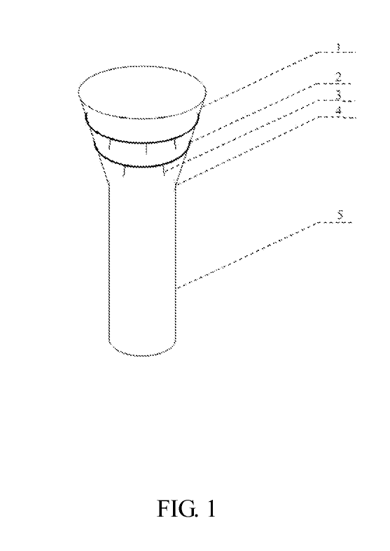

The elements or parts marked in FIG. 1 are an ampulla 1, a stretch yarn 2, an anchor hook 3, an acute angle 4, and a tubular portion 5.

### DETAILED DESCRIPTION OF THE INVENTION

In the following, the invention may be further described by combining the figure and specific example.

As shown in FIG. 1, the invention provides a duodenum endothelium membrane. The duodenum endothelium membrane can be divided into an ampulla 1 and a tubular portion 5. The ampulla 1 is the part of the trumpet-shaped consecutive tubular portion 5, as well columnar, spherical columnar and drum-shaped. The thickness of the endothelium membrane of the ampulla 1 is 0.005 mm-1 mm, and its height is 6 mm-100 mm. The trumpet-shaped consecutive tubular portion 5 is a progressive open acute angle 4, of which the angle is 5 degrees to 45 degrees. The thickness, height and angle of the ampulla 1 are matched with the different crowds. The diameter and length of the tubular portion 5 are matched with the duodenum and jejunum of different crowds, of which the diameter is 10-60 mm, and the length can be extended to a section of jejunum continued with duodenum. The length is 80-700 mm, and the endothelium membrane of the tubular portion 5 has thickness of 0.005 mm-1 mm. The tubular portion 5 is made of the elastic shape memory material. Therefore, when the intestinal peristalsis is transferred, the problem on the deflation of the tubular portion induced by gastrointestinal peristalsis and gap increase between the endothelium membrane of the tubular portion and the inner wall of the duodenum and then the regurgitation of jejunum contents can be avoided.

The stretch yarn 2 provided with the anchor hook is surrounded outside the ampulla 1 of consecutive tubular portion 5, of which the yarn diameter is 0.05 mm-1 mm; and the stretch yarn 2 and ampulla 1 are integrated as a whole. The explanate stretch yarn 2 can be rectilinear, as well V-shaped, U-shaped, O-shaped, grid-shaped and parabolic. The explanate stretch yarn 2 with various shapes can be surrounded the ampulla 1 both with the single circle or many circles, and each circle of multi-circle surrounding can be separated without closing. The stretch yarn 2 is attached with an anchor hook 3 every a distance, of which the distance is 5 mm-20 mm; and the anchor hook 3 is faced towards the end of the tubular portion 5, of which the included angle is 5 degrees-75 degrees, and the length is 0.3 mm-1 mm.

The duodenum endothelium membrane is soft, smooth, elastic, and has good histocompatibility, thereby, weakening the host response generated by implanting in the body. After being implanted in the body, the duodenum endothelium membrane can be gradually degraded through two months to five years in the body, and the degradation product and soluble substance are safe to the human body, and free of acute systemic response, chronic systemic response, acute local response and chronic local response, and has good histocompatibility, thereby, avoiding the organ tissue injury when removing from the body, and slowing down the “rebound” effect after the complete demolition of the original barrier in an instant by gradually degrading.

The ampulla 1 and tubular portion 5 can be converged into a spherical or capsular form, or folded into a spherical or capsular form in vitro together.

The duodenum endothelium membrane can be sent into the duodenum through the upper digestive tract in the assistance of endoscope and X-ray fluoroscopy, and under the intra-intestinal memory temperature, the stretch yarn 2 outside the ampulla 1 is gradually unfolded, the ampulla 1 is closely stuck to the duodenal wall. The anchor hook 2 on the stretch yarn 2 surrounded outside the ampulla 1 is naturally inserted into the intestinal mucosa of duodenal bulb cavity section and tela submucosa, so that the ampulla 1 of the duodenum endothelium membrane is solidly fixed at the duodenal bulb. The tubular portion 5 of the endothelium membrane is located at the back end of consecutive duodenal bulb of superior part of duodenum, and the extended tubular portion 5 is located at the ascending part of duodenum and consecutive jejunum section.

The duodenum endothelium membrane divides the chyme and bile-pancreatic juice in the body, so as to avoid the direct digestion, absorption and metabolism of gastric effluent in the duodenum. It is free of anaphylaxis, toxic response, anaphylaxis, mutagenicity, carcinogenicity, teratogenicity and inhibition of cell activation, and has good histocompatibility, thereby, weakening the host response generated by implanting in the body. After being implanted in the body, the duodenum endothelium membrane can be gradually degraded through two months to five years in the body, thereby, avoiding the organ tissue injury when removing from the body, and slowing down the “rebound” effect after the complete demolition of the original barrier in an instant by gradually degrading. The medical device prepared for treating the obesity and diabetes can prevent falling off, being free of removing, restraining bounce and reducing the injury.

### Embodiment One

A duodenum endothelium membrane, made of shape-memory thermosetting degradable biocompatible materials, mainly includes a tubular portion 5 and a trumpet-shaped consecutive ampulla 1 of which the outer side is surrounded a stretch yarn 2 provided with an anchor hook 3.

Taking pentaerythritol and 1, 1, 1-trimethylolethane as initiator and dibutyltin oxide (DBTO) as catalyst, and carrying out the ring opening polymerization on lactide and glycolide at 130° C., so as to obtain hydroxyl-terminated star prepolymer having different compositions, relative molecular mass and functionality. Then reacting with aliphatic diisocyanate at 80° C., so as to obtain the shape-memory thermosetting degradable biocompatible materials having the crosslinking structure.

The thickness for the endothelium membranes of the ampulla 1 and the tubular portion 5 is 0.005 mm-1 mm. The diameter and length of the tubular portion 5 are matched with the duodenum and jejunum in different crowds, of which the diameter is 10-60 mm, and the length can be extended to a section of the jejunum continued with the duodenum. The length is 80-700 mm. The height of the trumpet-shaped consecutive ampulla 1 is 6 mm-100 mm, and the trumpet-shaped consecutive tubular portion 5 is a progressive open acute angle 4, of which the angle is 5 degrees to 45 degrees. The thickness, height and angle of the ampulla 1 are matched with the different crowds. The ampulla 1 can also be columnar, spherical columnar, or drum-shaped.

The stretch yarn 2 is surrounded outside the ampulla 1, which can be surrounded both with the single circle or many circles, and each circle of multi-circle surrounding can be separated without closing. The stretch yarn 2 and ampulla 1 are integrated as a whole. The yarn diameter of the stretch yarn 2 is 0.05 mm-1 mm. The explanate stretch yarn 2 can be rectilinear, as well V-shaped, U-shaped, O-shaped, grid-shaped, or parabolic. The stretch yarn 2 is attached with an anchor hook 3 every 5 mm-20 mm, and the anchor hook 3, having the length of 0.3 mm-1 mm, is faced towards the end of the tubular portion, of which the included angle is 5 degrees-75 degrees.

The shape-memory degradable biocompatible materials surrounding the stretch yarn 2 outside the ampulla 1 can take polycaprolactone (PCL), polylactic acid (PLA), polyglycollide (PGA) and polyglycollide-lactide (PLGA), polytetramethylene ether glycol (PTMG) and copolymer thereof and other polyesters, having a good slow-release function and safe degradation product, as the soft segment, and take 2, 6-diisocyanate (LDI), 1, 4-diisocyanate (BDI), and hexamethylene diisocyanate (HDI) as well as copolymer thereof as the hard segment. Aromatic diisocyanate is reacted with polyester or polyether having certain molecular weight to generate prepolymer of carbamic acid, and then subjected to the chain extension with polyols or polyamine and others to generate polyurethane with a block structure. The shape-memory degradable biocompatible materials in the invention can be prepared with the different aggregation states and thermal behaviors of the soft segment (polyester or polyether) and the hard segment (carbamate) of the segmented polyurethane. The soft segment part is in a reversible phase, and when stretching at the certain heat degree, the curly molecular chain is stretched in the exogenic action and oriented, and then, cooled in the keeping of exogenic action. The soft segment is crystallized or vitrified to freeze the stress, and while the intrinsic heat at the certain temperature, the stress frozen is gradually released and gradually recovered the deformation in the effect of polyurethane elasticity. The hard segment part is a physical crosslinking point, and at certain heat degree, the deformation can be carried out in the interval above the melting point of the soft segment and below the melting point of the hard segment through giving the modulus and strength to the polyurethane, without breaking or permanent deformation (plastic deformation). Through adjusting the content or category of the soft segment and hard segment parts, the shape-memory degradable biocompatible materials having the required degradation rate, elasticity modulus, memory temperature, crystallinity, tensile strength, Young modulus and other main properties can be plasticized.

The ampulla 1 and tubular portion 5 can be converged into a spherical or capsular form, or folded into a spherical or capsular form in vitro together.

### Embodiment Two

A duodenum endothelium membrane, made of shape-memory thermosetting degradable biocompatible materials, mainly includes a tubular portion 5 and a trumpet-shaped consecutive ampulla 1 of which the outer side is surrounded a stretch yarn 2 provided with an anchor hook 3.

Synthesizing the shape-memory thermosetting degradable biocompatible materials by taking MDI, 1, 4-butanediola and poly-caprolaclone glycol as the raw materials with the prepolymerization method, carbamate is in the hard segment, and the formed physical crosslinking structure constitutes the stationary phase of the material. The poly-caprolaclone glycol constitutes the reversible phase of the material, of which the average relative molecular mass is 1600-8000. The transformation temperature of the material can be adjusted through changing the content of the poly-caprolaclone glycol and relative molecular mass.

The thickness for endothelium membranes of the ampulla 1 and tubular portion 5 is 0.005 mm-1mm. The diameter and length of the tubular portion 5 are matched with duodenum and jejunum in different crowds, of which the diameter is 10-60 mm, and the length can be extended to a section of the jejunum continued with the duodenum. The length is 80-700 mm. The height of the trumpet-shaped consecutive ampulla 1 is 6 mm-100 mm, and the trumpet-shaped consecutive tubular portion 5 is a progressive open acute angle 4, of which the angle is 5 degrees to 45 degrees. The thickness, height and angle of the ampulla 1 are matched with the different crowds. The ampulla can also be columnar, spherical columnar and drum-shaped.

The stretch yarn 2 is surrounded outside the ampulla 1, which can be surrounded both with the single circle and many circles, and each circle of multi-circle surrounding can be separated without closing. The stretch yarn 2 and ampulla 1 are integrated as a whole. The yarn diameter of the stretch yarn 2 is 0.05 mm-1 mm. The explanate stretch yarn 2 can be rectilinear, as well V-shaped, U-shaped, O-shaped, grid-shaped, and parabolic. The stretch yarn 2 is attached with an anchor hook 3 every 5 mm-20 mm, and the anchor hook 3, having the length of 0.3 mm-1 mm, is faced towards the end of the tubular portion 5, of which the included angle is 5 degrees-75 degrees.

The shape-memory degradable biocompatible materials surrounding the stretch yarn 2 outside the ampulla 1 can take polycaprolactone (PCL), polylactic acid (PLA), polyglycollide (PGA) and polyglycollide-lactide (PLGA), polytetramethylene ether glycol (PTMG) and copolymer thereof and other polyesters, having a good slow-release function and safe degradation product, as the soft segment, and take 2, 6-diisocyanate (LDI), 1, 4-diisocyanate (BDI), and hexamethylene diisocyanate (HDI) as well as copolymer thereof as the hard segment. Aromatic diisocyanate is reacted with polyester or polyether having certain molecular weight to generate prepolymer of carbamic acid, and then subjected to the chain extension with polyols or polyamine and others to generate polyurethane with a block structure. The shape-memory degradable biocompatible materials in the invention can be prepared with the different aggregation states and thermal behaviors of the soft segment (polyester or polyether) and hard segment (carbamate) of the segmented polyurethane. The soft segment part is in a reversible phase, and when stretching at certain heat degree, the curly molecular chain is stretched in the exogenic action and oriented, and then, cooled in the keeping of exogenic action. The soft segment is crystallized or vitrified to freeze the stress, and while the intrinsic heat at the certain temperature, the stress frozen is gradually released and gradually recovered the deformation in the effect of polyurethane elasticity. The hard segment part is a physical crosslinking point; and at certain heat degree, the deformation can be carried out in the interval above the melting point of the soft segment and below the melting point of the hard segment through giving the modulus and strength to the polyurethane, without breaking or permanent deformation (plastic deformation). Through adjusting the content or category of the soft segment and hard segment parts, the shape-memory degradable biocompatible materials having the required degradation rate, elasticity modulus, memory temperature, crystallinity, tensile strength, Young modulus and other main properties can be plasticized.

The ampulla 1 and tubular portion 5 can be converged into a spherical or capsular form, or folded into a spherical or capsular form in vitro together.

### Embodiment Three

### In Vitro Degradation Test

Sampling the massive and dry polymer, and putting the same in 0.1 mol/L NaOH water solution at 37±1° C. and imitated duodenal juice of which PH is 7.6 (replacing the degradation liquid every two weeks), taking out the sample, flush with distilled water, carrying out the vacuum drying on the sample at 40° C. and then weighing, and calculating the weight loss ratio. The degradation behavior for copolymer of polyester polyurethane (PE-U) at 37° C. shows that the proportion of PE-U3 with the strongest water absorbing capacity is in proportion to the degradation. However, the degradation speed of PE-U1 and PE-U2 is in inversely proportional to their water absorbing capacity, showing that the degradation behavior of PE-U copolymer is related to the water absorbing capacity of polymer and hydrolysis ability of ester bond on the each chain segment in the polymer, so as to set the degradation rate accordingly.

### Embodiment Four

### In Vivo Degradation Test

The degradation rate, elasticity modulus, crystallinity, tensile strength, Young modulus and other main properties can be controlled through designing and adjusting the proportion and category of the soft segment. Since the degradation behavior of polyurethane is decided by the degradation way of used prepolymer in the large extent, it can be controlled through adjusting the hydrolysis (such as copolymerizing some monomers) of prepolymer. The degradable polyurethane material can reserve the biodegradability of its prepolymer (aliphatic polyester, polyether ester, poly (ether ester amide) and others) to some extent, and can also change its mechanical property. Therefore, in this embodiment degradation is set for 3 months at least. The endothelium membrane is implanted in the duodenum of Guangxi Bama small xiang pig by uniting endoscope and X-ray. From the set third month, X-ray is used for observing, and the complete degradation is carried out after 17-23 days from then on.

### Embodiment Five

### Cytotoxicity Test

Taking the dermal stem cell suspension of a rabbit, of which the density is 6×10⁴/ml, transfusing the same in the culture dish, turning the culture dish so that the cell is uniformly dispersed on the surface, and culturing to the near confluence of single layer; discarding the culture dish, adding the fresh culture medium, putting the sample in the culture dish, and respectively dripping trypsin in the culture medium in the first, second, third, fourth, and sixth days so that the adherent cell on the wall of culture dish is fallen off to the culture medium, taking the quantitative culture medium to drip on the cell counting plate, and counting and evaluating the growth situation of cell; in the sixth day, observing the growth of dermal stem cell of the rabbit with inverted microscope in the culture dish. The cell of the experimental group is grown well in the culture medium of material edge, it can be seen that the cell has been attached at the material edge and grown in the culture medium of edge, and the cell is counted with the cell counting plate, of which the concentration is 8.7×10⁴/ml. Counting the cell in the culture dish in the first, second, third, fourth and sixth days, and drawing the cell growth curve, to prompt that the difference of cell growth between experimental group and normal control group has no remarkable significance (P<0.0.5).

### Embodiment Six

### Acute Systemic Toxicity Test

The leaching liquid of polyurethane material is prepared according to Part 12 of National standard-Biological evaluation of medical devices, and the specific operation step is as follows: taking the polar dielectric sterilized saline as the leaching medium, cutting the material into 0.6 cm×0.6 cm sheets, rinsing with tri-distilled water for three times, irradiating with cobalt-60 and then standby, the leaching proportion is 3 cm²/ml; and leaching the material for 72 h at 37° C. in the sterile state, so as to prepare the 100ml leaching liquid of polyurethane material; dividing 30 healthy male Balbc mice into three groups randomly, namely, leaching liquid group of polyurethane material, positive control group and negative control group; at the moment before test, weighing the each mouse, recording and marking; and shaking up the leaching liquid of polyurethane material, carrying out 50 ml/kg tail vein injection on Balbc mice, taking the normal saline as the negative control, taking the 4.5ml/L phenol water solution as the positive control, and observing the response and survival rate of Balbc mice within 72 h. The result shows that the mice in the polyurethane material group and negative control group have no death, free of adverse reaction have no remarkable significance on the difference of weight change, and meet with the standard, and the mice in the positive control group are diarrhea completely and the weight is reduced. Three Balbc mice have serious diarrhea, and the weight is less than 14 g.

### Embodiment Seven

### In Vivo Muscle Embedding Test

Cutting the material into 0.3cm×1.0 cm sheets, rinsing with tri-distilled water for three times, irradiating with cobalt-60 and then standby; dividing 12 healthy adult New Zealand rabbits of which the male and female are half and half into the three groups randomly, carrying out intraperitoneal injection anesthesia on 5 ml/kg 20% urethane, wiping out the hair in the operating zone on the back of rabbit, disinfecting the operating zone with povidone iodine, incising the skin, separating the subcutaneous tissue, and fully exposing the muscle beside the spine; embedding the material sheet along the long axis of muscle fiber at 30 mm away from the center line, sewing the skin, disinfecting, taking out stitches after 1 week, putting the animal to death without pain in one week, four weeks and twelve weeks, taking the muscular tissue beside the spine, cleaning up with the normal saline, cleaning with 4% paraformaldehyde, fixing the brain tissue in 4% paraformaldehyde solution, carrying out the paraffin embedding after 24 h, waxing, cutting into slices, carrying out HE staining, and observing with an optical microscope.

After the material is implanted for one week, cutting the animal skin at the embedding place, observing visually and showing that the animals in the material group have no obvious tissue edema, and carrying out the pathological section of HE staining to show that the edema of striated muscle is not obvious, the continuity of muscle fiber is good, there is little inflammatory cell infiltration in the interstitial tissue, and there is little fibrous tissue proliferation at the tissue edge of embedding place. After 12 weeks, the animals in the material group have no obvious tissue edema, the pathological section of HE staining shows that the tissue structure is complete, there is no obvious inflammatory response, and a few fibrous tissues are wrapped around the tissue at the embedding place.

### Embodiment Eight

### Allergic Test

Taking 4 g materials, adding 20 ml normal saline, and leaching for 1 h at 120° C.±2° C.; dividing 30 health guinea pigs of which the weight is 413.87±65.32 g and the male and female are not limited into three groups randomly, namely, material leaching liquid group, positive control group and negative control group. Sensitization: carrying out 0.5 ml q.o.d material leaching liquid of intraperitoneal injection on the guinea pigs in the material leaching liquid group for three times, injecting the calf serum on the guinea pigs in positive control group, and injecting the normal saline on the negative control group. Stimulation: carrying out 1.0 ml corresponding test solution of intraperitoneal injection on each guinea pig in 14 d, 21 d and 28 d after the first injection, continuously observing for 1 h, and finding that the guinea pigs in the experimental group and negative control group have no piloerection, dyspnea and other signs, and the guinea pigs in positive control group has piloerection, dyspnea and other signs, which prompts that the material has no sensitization.

### Embodiment Nine

### Skin Irritation Test

Dividing 12 chinchilla rabbits of which the weight is 3102±147 g into three groups randomly, taking 4% formaldehyde solution in the positive control group and normal saline in the negative control group, weighing 0.5 m material to wear into medium power, taking three unhairing areas with area of 2-3 cm at two sides of rabbit spine respectively, attaching the medium power, covering with four layers of gauzes, removing the object attached after binding up and fixing for 4 h, marking attaching part, cleaning with 75%(v/v) alcohol, and naturally drying at the room temperature. Scoring the attaching part respectively after 24 h, 48 h and 72 h, the evaluation standard and scoring are: 0 score for no erythema, 1 score for very weak erythema, 2 scores for clear light erythema, 3 scores for clear boundary and bright erythema, 4 scores for purple erythema with slight scab; 0 score for no edema, 1 score for very slight edema, 2 scores for mild edema (the edge is obviously higher than epidermis around), 3 scores for moderate edema (the edema is about 1 mm higher than epidermis around), 4 scores for serious edema (the edema is above 1 mm higher than epidermis around, and the area is more than attaching are); and no stimulation for 0-0.4 scores for two item, mild stimulation for 0.5-1.9 scores, moderate stimulation for 2.0-4.9 scores, and strong stimulation for 5.0 scores above. The result shows that: in the positive control group marking point, then three rabbits have mild erythema and edema after 24 h, three rabbits have moderate edema and erythema after 48 h, and three rabbits have serious edema and erythema and one rabbit has moderate edema and erythema after 72 h. For the material group, one support material marking point of one rabbit has slight erythema after 24 h, one support material marking point of one rabbit has slight erythema and edema, and the rabbits of other support material marking points have no remarkable significance on the difference with the negative control group, the erythema and edema are disappearance after 48 h, the marking points of material group have no remarkable significance on the difference with that of negative control group, and after 72 h, the difference between the marking points of material group and skin at the normal position has no remarkable significance.

### Embodiment Ten

### Mechanical Property (Elasticity) Test

Taking chitosan-polyurethane compounding as example, taking 100 mm×10 mm×0.5 mm of sample of material in size, testing with universal material testing machine, the gauge length is 50 mm, and the tensile speed is 10 mm/min. At the environment temperature of 25° C., testing for 5 times, the result shows that the stress-strain curve is basically kept original when the proportion of chitosan-polyurethane is 10:90 (namely, chitosan accounts for 10%, and polyurethane accounts for 90%), which can have enough mechanical property, and can also obtain the ideal hydroscopicity; and the elasticity modulus is increased somewhat near this point.

### Embodiment Eleven

### Therapeutic Effect to Diabetes

By referring to the diagnosis standard of human diabetes and combining the blood glucose of the animal in the embodiment, the fasting blood sugar of Guangxi Bama small xiang pig which is more than 8.50 mmol/L (generally higher than that of human) is risen abnormally.

Dividing 16 Guangxi Bama male small xiang pigs with 8 months old into three groups randomly according to the weight, namely, normal feed group (5 pigs), high-sugar and high-fat feed group (5 pigs), and high-sugar and high-fat feed and built-in duodenum endothelium membrane group (6 pigs). Insertion of the duodenum endothelium membrane: after anesthesia, the duodenum endothelium membrane is inserted into the duodenum by combining the endoscope and X-ray, and then designed for degrading from 3 months at least.

The formula of the high-sugar and high-fat feed is: 35% sucrose, 10% beef tallow, and 55% of basal feed, and the formula of basal feed is: 48% corn, 20% wheat middling, 15% soybean cake, 12% rice bran, and 5% fish meal. Feeding according to the field; feeding for 3 times every day, the daily ration is 4% of weight; and drinking water freely. Fasting overnight at the end of each month, then weighing, extracting the blood sample in the venous sinus, and determining the fasting blood-glucose, plasma triglyceride, free fatty acid and form and function of β-cell.

At the end of the first month, the fasting blood-glucose in the high-sugar and high-fat feed group is obviously increased to 8.94±0.73 mmol/L, and compared with the normal group, the difference has remarkable significance (P<0.01), the volume of β-cell is decreased, and the function of β-cell is reduced; and at the end of the second and third months (8.27±1.24 mmol/L, 9.05±0.68 mmol/L), compared with normal group, the difference still has remarkable significance (P<0.01). For the high-sugar and high-fat feed and built-in duodenum endothelium membrane group, the fasting blood-glucose is not obvious, 5.82±1.57 mmol/L, and compared with the normal group, the difference has no remarkable significance (P>0.05), the volume of β-cell is not decreased unobvious, and the function of β-cell is not reduced obviously; and at the end of the second and third months (5.15±0.62 mmol/L, 4.73±0.54 mmol/L), compared with the normal group, the difference still has no remarkable significance (P>0.05), which prompts that the duodenum endothelium membrane can be used for treating the diabetes and preventing the same.

The function of β-cell restores to period two or period one in tissues and functions.

At the end of the first month, the fasting weight in high-sugar and high-fat feed group is obviously increased to 54.78±7.41 kg, and compared with the normal group, the difference has remarkable significance (P<0.01); and at the end of the second and third months (57.23±6.27 kg, 61.13±8.35 kg), compared with the normal group, the difference still has remarkable significance (P<0.01). For the high-sugar and high-fat feed and built-in duodenum endothelium membrane group, the fasting weight is not obvious, 37.25±5.32 kg, and compared with the normal group, the difference has no remarkable significance (P>0.05); and at the end of the second and third months (39.51±6.27 kg, 41.86±5.74 kg), compared with the normal group, the difference still has no remarkable significance (P>0.05), which prompts that the duodenum endothelium membrane can be used for treating the obesity and preventing the same.

After being implanted in the body, the duodenum endothelium membrane is degraded at the set third month, and completely degraded after 18-22 days from then on by inspecting with X-ray. Compared with the normal group, the difference for fasting blood-glucose (4.33±0.41 mmol/L) of the high-sugar and high-fat feed and built-in duodenum endothelium membrane group at the end of the fourth month has no remarkable significance (P>0.05), and the difference of the fasting weight (38.74±6.32 kg) has no remarkable significance (P>0.05); and compare with the normal group, the difference for fasting blood-glucose (4.87±0.64 mmol/L) of the high-sugar and high-fat feed and built-in duodenum endothelium membrane group at the end of the fifth month still has no remarkable significance (P>0.05), and the difference of the fasting weight (40.84±6.96 kg) has no remarkable significance (P>0.05), which prompts that the original therapeutic outcome of diabetes and obesity has no “rebound” phenomenon after the duodenum endothelium membrane is gradually degraded in the body.

In the high-sugar and high-fat feed and built-in duodenum endothelium membrane group, putting one pig to death in the second month after the duodenum endothelium membrane is implanted in the body, taking out the duodenum immediately, fixing, waxing, embedding, and carrying out HE staining and pathological section, to show that the tissue structure of duodenum is complete, there is no obvious inflammatory response, but there are few fibrous tissues partially. In the high-sugar and high-fat feed and built-in duodenum endothelium membrane group, putting one pig to death in the fourth month after the duodenum endothelium membrane is implanted in the body, taking out the duodenum immediately, fixing, waxing, embedding, and carrying out HE staining and pathological section, to show that the tissue structure of duodenum is complete, there is no obvious inflammatory response, and the partial fibrous tissues are not obviously, which prompts that the duodenum endothelium membrane has good biocompatibility partially in the duodenum.

### Embodiment Twelve

### Therapeutic Action to Obesity

Dividing 10 Guangxi Bama male small xiang pigs with 7 months old into two groups randomly according to the weight, namely, non-embedded duodenum endothelium membrane group (5 pigs), built-in duodenum endothelium membrane (5 pigs). Two groups of pigs are excised ovary at two sides according to the minimally invasive ovariectomized method, and fed for 5 weeks after the estrogen level is reduced, which is a castration obesity model.

Insertion of the duodenum endothelium membrane: after feeding for 5 weeks, by carrying out anesthesia, the duodenum endothelium membrane is inserted into the duodenum by combining the endoscope and X-ray, and then designed for degrading from 3 months at least.

Feeding the basal feed, the formula of the basal feed is: 48% corn, 20% wheat middling, 15% soybean cake, 12% rice bran, and 5% fish meal; feeding according to the field; feeding for 3 times every day, the daily ration is 4% of weight; and drinking water freely; and fasting overnight at the end of each month, and then weighing.

At the end of the first month, the fasting weight in built-in duodenum endothelium membrane group is not obviously increased (39.93±6.73 kg), and compared with the non-embedded duodenum endothelium membrane group, the difference has remarkable significance (P<0.01); and at the end of the second and third months (37.15±7.14 kg, 34.49±6.27 kg), compared with the non-embedded duodenum endothelium membrane group, the difference still has remarkable significance (P<0.01), which prompts that the duodenum endothelium membrane can be used for treating the obesity and preventing the same.

After being implanted in the body, the duodenum endothelium membrane is degraded at the set third month, and completely degraded after 17-23 days from then on by inspecting with X-ray. Compared with the non-embedded duodenum endothelium membrane group, the difference for the fasting weight (35.68±7.92 kg) of the built-in duodenum endothelium membrane group at the end of the fourth month has no remarkable significance (P>0.05), and the difference of the fasting weight (36.44±6.52 kg) at the end of the fifth month still has no remarkable significance (P>0.05), which prompts that the original therapeutic outcome of diabetes and obesity has no “rebound” phenomenon after the duodenum endothelium membrane is gradually degraded in the body.

In the built-in duodenum endothelium membrane group, putting one pig to death in the fourth month after the duodenum endothelium membrane is implanted in the body, taking out the duodenum immediately, fixing, waxing, embedding, and carrying out HE staining and pathological section, to show that the tissue structure of duodenum is complete, there is no obvious inflammatory response, and the partial fibrous tissues are not obviously.

The parts including the same prior art or capable of being realized by the prior art are not involved in this invention.

1. (canceled)

2. (canceled)

3. A duodenum endothelium membrane, being made of degradable shape-memory biocompatible materials, comprising a soft, smooth, and elastic plastic model, wherein the model mainly includes an ampulla and a tubular portion, the tubular portion with the length is capable of extending to a jejunum, restricting food through the duodenum to weaken apoptosis of islet cells, the ampulla and the tubular portion can be converged or folded to a spherical or capsular form in vitro, the ampulla includes a stretch yarn with an anchor hook on its external encircle, thus to weaken a host response produced by implanting in vivo, during transfer of the bowel wall peristalsis, the tubular portion of the elastic plastic model prevents regurgitation of jejunum contents from both the deflation of the tubular portion such induced by gastrointestinal motility as well as the increase in gap space between the endothelium membrane of the tubular portion and an inner wall of the duodenum, and after being implanted in vivo, the duodenum endothelium membrane is gradually degraded in vivo through two months to five years, thus to avoid the complicated operation and organ tissue damage when removing from in vivo, to slow down the rebound effect when removing, and to prepare a medical device that prevents falling off, being free of removing, suppresses rebound, reduces damages, and treats diseases like obesity and diabetes.
